(19) United States
(12) Patent Application Publication
(10) Pub. No.: US 2022/0356486 A1
(43) Pub. Date: Nov. 10, 2022

(54) Tomato Spotted Wilt Virus Resistance in Cichorium

(71) Applicant: Bejo Zaden B.V., Warmenhuizen (NL)

(72) Inventors: Adriana Dorien Haarsma, Warmenhuizen (NL); Nicolaas Anthonius Zutt, Warmenhuizen (NL); Roberto Zattoni, Warmenhuizen (NL); Albertus Johannes Maria Schrijver, Warmenhuizen (NL)

(21) Appl. No.: 17/624,056

(22) PCT Filed: Jul. 5, 2019
(86) PCT No.: PCT/EP2019/068153

§ 371 Date: Dec. 30, 2021

(51) Int. Cl. C12N 15/82; C12Q 1/6895

(52) U.S. Cl. CPC C12N 15/8283; C12Q 1/6895; C12Q 2600/13

(57) ABSTRACT

The present invention relates to Tomato spotted wilt virus, or TSWV, resistant plants and especially plants of the genus Cichorium such as leaf chicory, endive, radicchio, Belgian endive, French endive, and witloof. The invention further relates to methods for identifying and providing the present plants using genomic nucleic acid sequences identified herein. Specifically, the present invention relates to plants being resistant to Tomato spotted wilt virus, or TSWV, wherein the plants comprise a first resistance providing genomic fragment comprising one or more nucleic acid sequences selected from the group consisting of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, and SEQ ID No. 26.

### CROSS-REFERENCE TO RELATED APPLICATION

This application is the United States national phase of International Application No. PCT/EP2019/068153 filed Jul. 5, 2019, the disclosure of which is hereby incorporated by reference in its entirety.

### SEQUENCE LISTING

The Sequence Listing associated with this application is filed in electronic format via EFS-Web and is hereby incorporated by reference into the specification in its entirety. The name of the text file containing the Sequence Listing is 9704-2108128_ST25.txt. The size of the text file is 19,939 bytes, and the text file was created on Dec. 16, 2021.

### SUMMARY

The present invention relates to Tomato spotted wilt virus, or TSWV, resistant plants and especially plants of the genus Cichorium such as leaf chicory, endive, radicchio, Belgian endive, French endive, and witloof. The invention further relates to methods for identifying and providing the present plants using genomic nucleic acid sequences identified herein.

Tomato spotted wilt virus (TSWV) is a spherical, negative-sense RNA virus with a diameter between 80 to 110 nm. The virus is transmitted by at least ten different species of thrips where the western flower thrips, or Frankliniella occidentalis, is the most prominent vector. Infection with TSWV causes serious losses in several economically important crops among which Cichorium species.

The virus is transmitted globally in the field but also in greenhouses. The high reproductive rate of thrips contributes to the spread of the virus. The amount of time it takes for F. occidentalis larvae to acquire a virus (acquisition period) and the amount of time it takes for the virus to move from the insect to the plant (inoculation) for TSWV can be as short as only 5 minutes. However, the acquisition and inoculation periods for optimal transmission are 21.3 hours and 42.7 hours, respectively.

Adult thrips cannot be infected with TSWV as their midgut barrier successfully prevents infection. However, thrips that have become infected with TSWV in the larval stage can transmit the virus throughout their lifetime.

In order to protect their eggs, thrips insert their eggs into various types of plant tissue. Therefore, eggs can be found in the stems, leaves, or flowers of plants. Adult thrips feed on the flower bud, stem and leaf parts of the plant.

More than 230 species in over 130 genera are known to be susceptible to TSWV. The large number of species illustrates the fact that economically TSWV is one of the most devastating plant viruses in the world.

In agriculture and horticulture, the main approach of managing TSWV is by prevention. An infected plant cannot be cured from the virus and is a source of further infections in a field or greenhouse. Measures taken for prevention include application of thrips- and virus free material, application of biological control of thrips by introduction of predators like the bug Orius insidiosus and Geocoris punctipes; removing of weed and infected plants and removal and destruction of old crops.

Also transport is a way of spreading the virus; in a factsheet from the Colorado University, consequences of the rapid spread caused by transport of TSWV in the US greenhouse industry is described ref. 4.

However, the above measures are time consuming, laborious and expensive. Accordingly, there is a need in the art to provide plants that encompass genetically encoded resistance.

Cichorium is a genus of plants in the dandelion tribe within the sunflower family. The genus includes cultivated species commonly known as chicory or endive, plus several wild species. Common chicory (Cichorium intybus) is a bushy perennial herb with generally blue or lavender flowers. It grows as a wild plant on roadsides in Europe where it is native, and in North America, where it has become naturalized. It is generally cultivated for its leaves such as leaf chicory, endive, radicchio, Belgian endive, French endive, or witloof. Other varieties are grown for their taproots, which are used to produce a coffee substitute, similar to dandelion coffee. Genetic resistance is, next to prevention and hygienic measures, the most efficient and economical way to protect Cichorium plants from diseases.

The symptoms of TSWV vary from host to host and symptoms within a single type of host are variable due to the age of the plant, nutrition and the environment (especially temperature). The most observed symptoms include stunting, ringspots on fruit and necrosis of leaves. Furthermore, there are many different strains of TSWV, differences in symptoms may also be attributed to the differences in the number of strains present.

An infection of Cichorium generally starts with bright spots in the affected leaves; in an advanced stage ring-shaped pigment deposits can be observed and tissue in these spots dies and finally, the complete plant has died due to the virus infection.

Some resistance genes are effective and very durable while other genes, e.g. Sw-5 in tomato, can be overcome by certain strains of TSWV. Many resistance genes act through a hypersensitive response. A hypersensitive response is characterized by the fact that plant cells surrounding the infected area undergo cell death, thus isolating the virus and depriving it from the cells it needs for replication and further infection. However, in several countries, multiple TSWV strains are detected that can overcome the aforementioned Sw-5 resistance gene.

The symptoms of TSWV vary from host to host and symptoms within a single type of host are variable due to the age of the plant, nutrition and the environment (especially temperature). The most observed symptoms include stunting, ringspots on fruit and necrosis of leaves. Furthermore, there are many different strains of TSWV, differences in symptoms may also be attributed to the differences in the number of strains present. An infection, of Cichorium generally starts with bright spots in the affected leaves; in an advanced stage ring-shaped pigment deposits can be observed and tissue in these spots dies and finally, the complete plant has died due to the virus infection.

### BRIEF DESCRIPTION OF DRAWINGS

FIG. 1 shows a representation of linkage group 5 (maximum LOD=20), i.e. the present first resistance providing genomic fragment, of Cichorium for TSWV resistance.

FIG. 2 shows a representation of linkage group 1 (maximum LOD=3.84), i.e. the present second resistance providing genomic fragment, of Cichorium for TSWV resistance.

### DETAILED DESCRIPTION

It is an object of the present invention, amongst other objects, to address the above identified need in the art.

This object of the present invention, amongst other objects, is met by providing plants, methods and nucleic acid sequences outlined in the appended claims.

Specifically this object of the present invention, amongst other objects, is met by providing plants being resistant to Tomato spotted wilt virus, or TSWV, wherein the plant comprises a first resistance providing genomic fragment comprising one or more nucleic acid sequences selected from the group consisting of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, and SEQ ID No. 26.

The present inventors surprisingly discovered that a genomic region on linkage group 5 at approximately 100 to 108 cM, more specifically between 101 to 102 cM, such as 102.7 cM, was capable of providing Tomato spotted wilt virus, or TSWV, resistance. The present inventors also discovered that this resistance could be further enhanced if the discovered genomic region was combined with a second genomic region.

Accordingly, the present invention preferably relates to plant further comprising a second resistance providing genomic fragment on linkage group 1 comprising one or more nucleic acid sequences selected from the group consisting of SEQ ID No. 28, SEQ ID No. 30, SEQ ID No. 32, SEQ ID No. 34, SEQ ID No. 36, SEQ ID No. 38, SEQ ID No. 40, SEQ ID No. 42, SEQ ID No. 44, SEQ ID No. 46, SEQ ID No. 48, SEQ ID No. 50, and SEQ ID No. 52.

According to the present invention, the present first genomic fragment preferably comprises SEQ ID No. 14 and the second genomic fragment comprises SEQ ID No. 36, SEQ ID No. 38, SEQ ID No. 40 and/or SEQ ID No. 42.

The present plants are preferably plants of the genus Cichorium, more preferably leaf chicory, endive, radicchio, Belgian endive, French endive, and witloof.

Both the present first and second resistance providing genomic fragment are present in a Cichorium plant of which representative seed are deposited on Mar. 13, 2019, as NCIMB 43371 (Ferguson Building, Craibstone Estate, Bucksburn, Aberdeen AB21 9YA, United Kingdom).

According, the present first and second resistance providing genomic fragments are preferably obtainable, obtained or derived from a Cichorium plant of which representative seed are deposited under NCIMB 43371.

According to an especially preferred embodiment, the present plants are hybrid plants. Generally, hybrids plants result from the cross between two parent plants producing an F1 hybrid (first filial generation). The cross between the two parents lines produces F1 hybrids which are phenotypically homogeneous. Within the context of the present invention, also double cross hybrids and three-way cross hybrids are contemplated although single cross hybrids are preferred.

Considering the beneficial properties of the presently identified resistance, the present invention relates to method for identifying a plant being resistant to Tomato spotted wilt virus, or TSWV, the methods comprise the step of establishing the presence of first resistance providing genomic fragment comprising one or more nucleic acid sequences selected from the group consisting of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, and SEQ ID No. 26 in the genome of the plant.

Preferably, the present methods further comprise the step of establishing the presence of a second resistance providing genomic fragment comprising one or more nucleic acid sequences selected from the group consisting of SEQ ID No. 28, SEQ ID No. 30, SEQ ID No. 32, SEQ ID No. 34, SEQ ID No. 36, SEQ ID No. 38, SEQ ID No. 40, SEQ ID No. 42, SEQ ID No. 44, SEQ ID No. 46, SEQ ID No. 48, SEQ ID No. 50, and SEQ ID No. 52 in the genome of the plant.

The present invention also relates to the use of one or more of nucleic acid sequences selected from the group consisting SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28, SEQ ID No. 30, SEQ ID No. 32, SEQ ID No. 34, SEQ ID No. 36, SEQ ID No. 38, SEQ ID No. 40, SEQ ID No. 42, SEQ ID No. 44, SEQ ID No. 46, SEQ ID No. 48, SEQ ID No. 50, and SEQ ID No. 52 for identifying or providing a plant being resistant to Tomato spotted wilt virus, or TSWV.

Further, the present invention relates to methods for providing a plant being resistant to Tomato spotted wilt virus, or TSWV, wherein the method comprises introgressing a first, or a first and second, genomic fragment obtainable, obtained or derived from a Cichorium plant of which representative seed are deposited under NCIMB 43371 into the plant.

Furthermore, the present invention relates to nucleic acid sequence selected from the group consisting of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28, SEQ ID No. 30, SEQ ID No. 32, SEQ ID No. 34, SEQ ID No. 36, SEQ ID No. 38, SEQ ID No. 40, SEQ ID No. 42, SEQ ID No. 44, SEQ ID No. 46, SEQ ID No. 48, SEQ ID No. 50, and SEQ ID No. 52.

The present invention will be further detailed in the following examples. In the examples, reference is made to figures wherein:

### EXAMPLES

### Summary

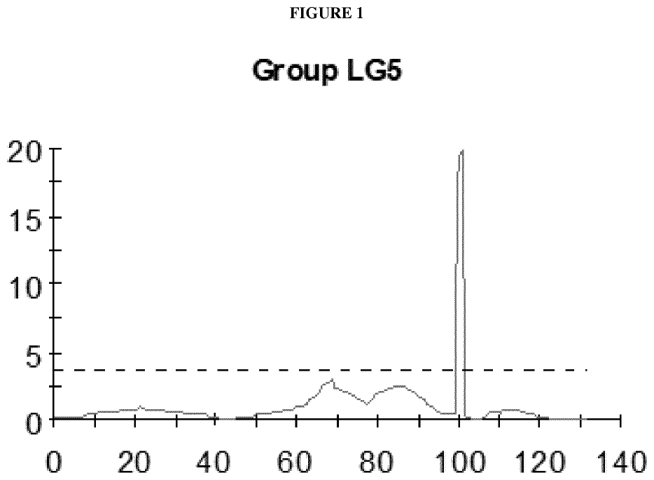

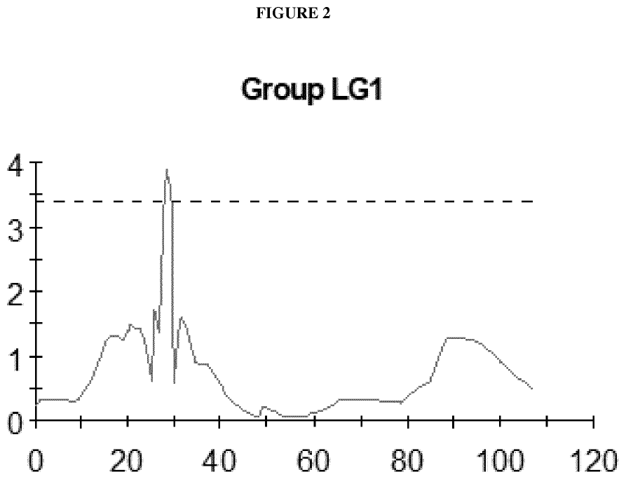

The presented disease scores in chicory assessments originate from TSWV trials in areas where the virus is already present in the field. A self-pollinated population of a hybrid between resistant and susceptible plants (F1S1) was chosen to perform disease trials. Individual plants were sampled and the phenotypes (susceptible vs. resistant) were scored. QTL analysis showed two QTLs, one major QTL at LG5 (FIG. 1) and a minor QTL on LG1 (FIG. 2). The presence of QTL on LG5 alone results in plants scoring a 7 on a scale of 0 (susceptible) to 9 (resistant). Presence of both QTL's results in a level 9 on this scale. The inheritance observed for both QTL's is codominant.

### Example 1: Field Testing for Resistance Against TSWV

To test breeding material ideal locations with the right climatic conditions to perform a TSWV field trial were identified. TSWV infected thrips are endemic in these areas and therefore pose a high disease pressure on the crop. By performing the test in this area, infection of the plants with TSWV is certain.

Cichorium intybus var. foliosum (radicchio rosso) plants to be tested were planted in the field. A radicchio variety that is susceptible to TSWV was used to increase the disease pressure in the field and is also the negative control. Plants were assessed for virus symptoms 75 days after transplanting of seedlings in the field. The scoring of the symptoms of virus infection is represented on a scale ranging from 1 (susceptible) to 9 (resistant).

### Example 2: Results from Field Tests for Level of TSWV Resistance in Cichorium Material

Material from the resistance source, a susceptible plant and the deposit (hybrid) were tested on two locations as described in example 1, where natural infection with TSWV occurs. The results are summarized in Table 1 below.

TABLE 1
Results field tests Cichorium

| | | Score | Score |
|---|---|---|---|
| Fieldnumber | description | location Fu3^{1, 3} | location Gu2^{2, 3} |
| Z10214 | Deposit NCIMB43371 | 7 | 7 |
| Z10225 | susceptible | 3 | 1 |
| Z19121 | resistant | 9 | 9 |

¹Italy
²Guatemala
³scores are between 1 (=very susceptible) and 9 = resistant

### Example 3: Molecular Characterization of Genomic DNA and Mapping of the Resistance Genes

Applying the available genetic resource for resistance as deposited at NCIMB (as NCIMB 43371), a F1S1 population was made by crossing the source material of resistance with a susceptible chicory line, after which the resulting F1 plant was self-pollinated. This population was chosen to perform a disease trial in naturally infected fields. Individual plants were sampled and the phenotype was scored.

Based on earlier research, a partial genetic map of Cichorium was constructed. Using SNP markers covering the entire genome, resistance loci were determined to be located on linkage groups 1 and 5. Both QTL's on the linkage groups showed a dominant inheritance.

For genetic linkage mapping, the Illumina Infinium platform was used for sequencing several parent lines of Cichorium intybus and Cichorium endive. The sequence information was used to identify a large set of SNP's which were used to genotype a mapping population which resulted in a general genetic map for Cichorium. Informative SNP's, well distributed along the entire Cichorium genome, were selected based on informativity between the resistant and susceptible chicory line and subsequently used for genotyping the phenotyped F1 S1 mapping population.

A genetic map was made using JoinMap 4.1 and the QTL-analysis was performed with MapQTL 6 (https://www.kyazma.nl (2017)). Through the MQM Mapping analysis, two QTL regions were identified, one major QTL region was found on linkage group 5 with a LOD score of 20; the second QTL was identified on linkage group 1, with a LOD score of 3.84.

### Deposit Information:

NCIMB 43371 deposited on Mar. 13, 2019 at NCIMB Limited, Ferguson Building; Craibstone Estate, Bucksburn ABERDEEN, Scotland, AB21 9YA United Kingdom.

### Abbreviations:

nm nanometer (10⁻⁹ m)
LG: linkage group
cM: centimorgans
SNP single nucleotide polymorphism
TSWV: Tomato spotted wilt virus
LOD score: Log of the Odds ratio. This unit stands for the likelihood two markers are linked divided by the likelihood that they are not linked; here a LOD score of 3,4 or more indicates that loci are close to the trait of interest.

TABLE 2
Genomic sequences

| | | | Position (cM) |
|---|---|---|---|
| SeqID | code | LG | [symbol] |
| 1 | 4059S | LG5 | 100.6 |
| 2 | 4059R | LG5 | 100.6 |
| 3 | 4056S | LG5 | 100.7 |
| 4 | 4056R | LG5 | 100.7 |
| 5 | 4085S | LG5 | 100.9 |
| 6 | 4085R | LG5 | 100.9 |
| 7 | 4063S | LG5 | 101.0 |
| 8 | 4063R | LG5 | 101.0 |
| 9 | 4067S | LG5 | 101.3 |
| 10 | 4067R | LG5 | 101.3 |
| 11 | 4088S | LG5 | 101.8 |
| 12 | 4088R | LG5 | 101.8 |
| 13 | 4081S | LG5 | 102.7 |
| 14 | 4081R | LG5 | 102.7 |
| 15 | 4068S | LG5 | 105.1 |
| 16 | 4068R | LG5 | 105.1 |
| 17 | 4084S | LG5 | 106.8 |
| 18 | 4084R | LG5 | 106.8 |
| 19 | 4064S | LG5 | 106.9 |
| 20 | 4064R | LG5 | 106.9 |
| 21 | 4083S | LG5 | 107.0 |
| 22 | 4083R | LG5 | 107.0 |
| 23 | 4061S | LG5 | 107.3 |
| 24 | 4061R | LG5 | 107.3 |
| 25 | 4089S | LG5 | 107.6 |
| 26 | 4089R | LG5 | 107.6 |
| 27 | 3837S | LG1 | 26.4 |
| 28 | 3837R | LG1 | 26.4 |
| 29 | 3853S | LG1 | 27.4 |
| 30 | 3853R | LG1 | 27.4 |
| 31 | 3905S | LG1 | 27.4 |
| 32 | 3905R | LG1 | 27.4 |
| 33 | 4022S | LG1 | 27.9 |
| 34 | 4022R | LG1 | 27.9 |
| 35 | 3891S | LG1 | 28.0 |
| 36 | 3891R | LG1 | 28.0 |
| 37 | 3935S | LG1 | 28.5 |
| 38 | 3935R | LG1 | 28.5 |
| 39 | 3957S | LG1 | 28.7 |
| 40 | 3957R | LG1 | 28.7 |
| 41 | 3921S | LG1 | 29.0 |
| 42 | 3921R | LG1 | 29.0 |
| 43 | 3856S | LG1 | 29.1 |
| 44 | 3856R | LG1 | 29.1 |
| 45 | 3865S | LG1 | 30.8 |
| 46 | 3865R | LG1 | 30.8 |
| 47 | 3849S | LG1 | 32.8 |
| 48 | 3849R | LG1 | 32.8 |
| 49 | 4010S | LG1 | 33.4 |
| 50 | 4010R | LG1 | 33.4 |
| 51 | 3960S | LG1 | 34.2 |
| 52 | 3960R | LG1 | 34.2 |

[symbol]

indicates data missing or illegible when filed

Genomic sequences in bold have the highest LOD score
SNP sequences are given in pairs:
the odd line numbers represent the sequence corresponding to the susceptible allele (code ending on S)
the even numbers represent the sequences corresponding to the resistant allele (code ending on R

TABLE 3
Abbreviations are according to IUPAC nucleotide code

| | | | |
|---|---|---|---|
| A = adenine | C = cytosine | G = guanine | T = thymine |
| R = A or G | Y = C or T | S = G or C | W = A or T |
| K = G or T | M = A or C | B = C or G or T | D = A or G or T |
| H = A or C or T | V = A or C or G | N = any base | . or - = gap |

1. A plant resistant to Tomato spotted wilt virus (TSWV), the plant comprising a first resistance providing genomic fragment having one or more nucleic acid sequences selected from the group consisting of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, and SEQ ID No. 26.

2. The plant according to claim 1, wherein the first resistance providing genomic fragment is the nucleic acid sequence of SEQ ID No. 14.

3. The plant according to claim 1, wherein the plant further comprises a second resistance providing genomic fragment having one or more nucleic acid sequences selected from the group consisting of SEQ ID No. 28, SEQ ID No. 30, SEQ ID No. 32, SEQ ID No. 34, SEQ ID No. 36, SEQ ID No. 38, SEQ ID No. 40, SEQ ID No. 42, SEQ ID No. 44, SEQ ID No. 46, SEQ ID No. 48, SEQ ID No. 50, and SEQ ID No. 52.

4. The plant according to claim 3, wherein the second resistance providing genomic fragment is the nucleic acid sequence of SEQ ID No. 36, SEQ ID No. 38, SEQ ID No. 40 SEQ ID No. 42.

5. The plant according to claim 1, wherein the plant is of the genus Cichorium.

6. The plant according to claim 5, wherein the plant is selected from the group consisting of leaf chicory, endive, radicchio, Belgian endive, French endive, and witloof.

7. The plant according to claim 1, wherein the first resistance providing genomic fragment and a second resistance providing genomic fragment are obtained from a Cichorium plant of which representative seed is deposited under NCIMB 43371.

8. The plant according to claim 1, wherein the plant is a hybrid plant.

9. A method for identifying a plant being resistant to Tomato spotted wilt virus (TSWV), the method comprising the step of establishing the presence of a first resistance providing genomic fragment having one or more nucleic acid sequences selected from the group consisting of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, and SEQ ID No. 26 in the genome of the plant.

10. The method according to claim 9, the method further comprising the step of establishing the presence of a second resistance providing genomic fragment having one or more nucleic acid sequences selected from the group consisting of SEQ ID No. 28, SEQ ID No. 30, SEQ ID No. 32, SEQ ID No. 34, SEQ ID No. 36, SEQ ID No. 38, SEQ ID No. 40, SEQ ID No. 42, SEQ ID No. 44, SEQ ID No. 46, SEQ ID No. 48, SEQ ID No. 50, and SEQ ID No. 52 in the genome of the plant.

11. (canceled)

12. A method for providing a plant being resistant to Tomato spotted wilt virus (TSWV), wherein the method comprises introgressing a first, or a first and a second, genomic fragment obtained from a Cichorium plant of which representative seed are deposited under NCIMB 43371 into the plant.

13. (canceled)


SEQUENCE LISTING (52 sequences)

SEQ ID NO: 1 - DNA, 201 bp, Cichorium intybus
taaaataatatttataataataataagttttcttattcgcttcaaaccctaaaaaaaacccatcggcaacattcatccaccacaagccaaaactgctagaaacagaacaacagatttctcgggttgccaacaacaaaataaaaccacaaaacatttacaattcaccgdtttataaaaacccccccatgtgggccccacwtc

SEQ ID NO: 2 - DNA, 201 bp, Cichorium intybus
taaaataatatttataataataataagttttcttattcgcttcaaaccctaaaaaaaacccatcggcaacattcatccaccacaagccaaaactgctagacacagaacaacagatttctcgggttgccaacaacaaaataaaaccacaaaacatttacaattcaccgdtttataaaaacccccccatgtgggccccacwtc

SEQ ID NO: 3 - DNA, 201 bp, Cichorium intybus
ctgyccwtgaacgyggtcaaggagastcaagtttctcagttgcaggtcctgtttcagggctcataacttactccgggcagatagctttttcgggcagcgtatctatccggtcagatagcagtacaacwagcaccagatccttygcctttcccatactgcagacagagtggaacagcagcccggtcaggatggcgaaagctg

SEQ ID NO: 4 - DNA, 201 bp, Cichorium intybus
ctgyccwtgaacgyggtcaaggagastcaagtttctcagttgcaggtcctgtttcagggctcataacttactccgggcagatagctttttcgggcagcgtctctatccggtcagatagcagtacaacwagcaccagatccttygcctttcccatactgcagacagagtggaacagcagcccggtcaggatggcgaaagctg

SEQ ID NO: 5 - DNA, 201 bp, Cichorium intybus
caccactaacgtcaccatttcttaatttcctatttccaccatcactaaactcaaatcacacactcaacctgattccaataagaaattaagaaatggtgacattagtggtgtcaacaacsaccgatccggcytcktacggmccggcrtcagcgctctccgccttggggaattggaaccccggcgtatcmtttgaggggatca

SEQ ID NO: 6 - DNA, 201 bp, Cichorium intybus
caccactaacgtcaccatttcttaatttcctatttccaccatcactaaactcaaatcacacactcaacctgattccaataagaaattaagaaatggtgacgttagtggtgtcaacaacsaccgatccggcytcktacggmccggcrtcagcgctctccgccttggggaattggaaccccggcgtatcmtttgaggggatca

SEQ ID NO: 7 - DNA, 201 bp, Cichorium intybus
acacwcgcgccgccttcatagtccaagtcaccgacaacgactcagggctagcggtaaccgaaccggaaagactcgccgtcataaaacaaatgcttagcaacatactcgaaggtagcaayaargctcgagaggcsacracggtggtctcccatggcgtaatccacaccgagcggcgcctccaccagatgatgctcgcggatc

SEQ ID NO: 8 - DNA, 201 bp, Cichorium intybus
acacwcgcgccgccttcatagtccaagtcaccgacaacgactcagggctagcggtaaccgaaccggaaagactcgccgtcataaaacaaatgcttagcaatatactcgaaggtagcaayaargctcgagaggcsacracggtggtctcccatggcgtaatccacaccgagcggcgcctccaccagatgatgctcgcggatc

SEQ ID NO: 9 - DNA, 201 bp, Cichorium intybus
ggatatacgaagacggsaaacacacttgtccggtcaccggcgtgaacttgaccaccctcgagcctgtaccyaaccacacgatcagaaagatgatacaaaactggtgcgtcgagaacaagtcatatgggtttgaycggatyccracgcctcgagctccggcgagctcgtatcaaatctccgacattctatccaagattatcg

SEQ ID NO: 10 - DNA, 201 bp, Cichorium intybus
ggatatacgaagacggsaaacacacttgtccggtcaccggcgtgaacttgaccaccctcgagcctgtaccyaaccacacgatcagaaagatgatacaaaattggtgcgtcgagaacaagtcatatgggtttgaycggatyccracgcctcgagctccggcgagctcgtatcaaatctccgacattctatccaagattatcg

SEQ ID NO: 11 - DNA, 201 bp, Cichorium intybus
aagtaataacmacagatcatcatggygattacagagcttccmtyaaactctctcttactcctggwtcvatgcttgtaatggaaggaaaatccgccgattacgctaaacaygcaataccctcaatccgraaacaamgaatwcttgtaacgttcgccaagtcactaccaaagcagtgggycccaccgccaaagcactatgtcc

SEQ ID NO: 12 - DNA, 201 bp, Cichorium intybus
aagtaataacmacagatcatcatggygattacagagcttccmtyaaactctctcttactcctggwtcvatgcttgtaatggaaggaaaatccgccgattatgctaaacaygcaataccctcaatccgraaacaamgaatwcttgtaacgttcgccaagtcactaccaaagcagtgggycccaccgccaaagcactatgtcc

SEQ ID NO: 13 - DNA, 201 bp, Cichorium intybus
actcgttatcattacgtgrgcccckatastctcttccytcrtttattctatctctaatcctgccatgtggcggtctaggagagtatctygaccgattccgccttcccatgtcaatttcccgttctaccctcgagtcgttctcrttgccacctggcatctttattctccctctaagccggctactaagbgatccggtatcca

SEQ ID NO: 14 - DNA, 201 bp, Cichorium intybus
actcgttatcattacgtgrgcccckatastctcttccytcrtttattctatctctaatcctgccatgtggcggtctaggagagtatctygaccgattccgtcttcccatgtcaatttcccgttctaccctcgagtcgttctcrttgccacctggcatctttattctccctctaagccggctactaagbgatccggtatcca

SEQ ID NO: 15 - DNA, 201 bp, Cichorium intybus
agatgacttaacccctcaaaatccggytttttgtcyctaaaawtatgtaaaacttcatctctagcacgakcttgccaattcgtgtgttgacccaataaaaccatagtccaaatcaacaaatctcttgtagtttcttgtccagcaacataaaagagcttgcattcctcrataatttcctccatacttarcccrgagttttta

SEQ ID NO: 16 - DNA, 201 bp, Cichorium intybus
agatgacttaacccctcaaaatccggytttttgtcyctaaaawtatgtaaaacttcatctctagcacgakcttgccaattcgtgtgttgacccaataaaatcatagtccaaatcaacaaatctcttgtagtttcttgtccagcaacataaaagagcttgcattcctcrataatttcctccatacttarcccrgagttttta

SEQ ID NO: 17 - DNA, 201 bp, Cichorium intybus
tgtacagaatctctggtagaattcgactatgacaacagtggtgatagatttaattccrttgatccacgtggcaagattcgttttataaaagtsaaycgtagtcaacgacattctcctaaaaaaattaccaagtctgcccctcgaccaccaccaccgccgccaataccaccgccaccaatacyaccgccaccacaactacaa

SEQ ID NO: 18 - DNA, 201 bp, Cichorium intybus
tgtacagaatctctggtagaattcgactatgacaacagtggtgatagatttaattccrttgatccacgtggcaagattcgttttataaaagtsaaycgtattcaacgacattctcctaaaaaaattaccaagtctgcccctcgaccaccaccaccgccgccaataccaccgccaccaatacyaccgccaccacaactacaa

SEQ ID NO: 19 - DNA, 201 bp, Cichorium intybus
ttcaataacttcaacctcatcttcactcggaattctccacccacgaccaccacayccaccacctcctccsccgccgcttatactctgtttcattgcggttatactgcaagtgaatcratatcgtttttcagacgaaaggattgaaaacccagttgcatatggcgawtaagagaatcagatatgagatttagagagaaagat

SEQ ID NO: 20 - DNA, 201 bp, Cichorium intybus
ttcaataacttcaacctcatcttcactcggaattctccacccacgaccaccacayccaccacctcctccsccgccgcttatactctgtttcattgcggttttactgcaagtgaatcratatcgtttttcagacgaaaggattgaaaacccagttgcatatggcgawtaagagaatcagatatgagatttagagagaaagat

SEQ ID NO: 21 - DNA, 201 bp, Cichorium intybus
cggttttaggagcaaagataaagaagatagggaacaaagaaaccgaagttgtgttggtgaaatggaagcatactcatgggcgaaatcgcacgtgggaggtcaargaggatatgaagaaaagataccctgattttgtagattatgatgcgattccsatgagrcagtcctcttaaggggtagataattgtaacatccttaaaa

SEQ ID NO: 22 - DNA, 201 bp, Cichorium intybus
cggttttaggagcaaagataaagaagatagggaacaaagaaaccgaagttgtgttggtgaaatggaagcatactcatgggcgaaatcgcacgtgggaggttaargaggatatgaagaaaagataccctgattttgtagattatgatgcgattccsatgagrcagtcctcttaaggggtagataattgtaacatccttaaaa

SEQ ID NO: 23 - DNA, 201 bp, Cichorium intybus
aggtgatgaagcggagaatgttggttccyggaatcagcaaaagtcaggaattcgtgatggtgaagaagaagaaatcsaaggtttttgcgtcgagtcgaagcatgggaagttcgccsaggagggaattagggtacgcgaatgcggatatgttggatgttttggatggattgggtacaagcttttaagctgttggtacatata

SEQ ID NO: 24 - DNA, 201 bp, Cichorium intybus
aggtgatgaagcggagaatgttggttccyggaatcagcaaaagtcaggaattcgtgatggtgaagaagaagaaatcsaaggtttttgcgtcgagtcgaagtatgggaagttcgccsaggagggaattagggtacgcgaatgcggatatgttggatgttttggatggattgggtacaagcttttaagctgttggtacatata

SEQ ID NO: 25 - DNA, 201 bp, Cichorium intybus
cggaagttgttgatgttgttgtggtcatggcggttgatctcttcttcctatcttcttcatactcaggatcrtctgtaggcaactcatacctacaaataggacawgtgttcctcgaacctaaccatggcactatacaatcttcatgatacatgtggccgcaaggcagttgcttcacaatctcctcctcattgttgaacactt

SEQ ID NO: 26 - DNA, 201 bp, Cichorium intybus
cggaagttgttgatgttgttgtggtcatggcggttgatctcttcttcctatcttcttcatactcaggatcrtctgtaggcaactcatacctacaaatagggcawgtgttcctcgaacctaaccatggcactatacaatcttcatgatacatgtggccgcaaggcagttgcttcacaatctcctcctcattgttgaacactt

SEQ ID NO: 27 - DNA, 201 bp, Cichorium intybus
caatgatctgctaacaattccggtaacccacacgatataaacartgttgaccaccacgatcagtgatgaaccgagtcaaagccaaactggttcgagcgtcagtaacaatggtagaaccaggatgctcctttaaracaatggcagccataagtgctatcaatttatcaccattaatggggttgccctctttgtcaacaacgc

SEQ ID NO: 28 - DNA, 201 bp, Cichorium intybus
caatgatctgctaacaattccggtaacccacacgatataaacartgttgaccaccacgatcagtgatgaaccgagtcaaagccaaactggttcgagcgtcggtaacaatggtagaaccaggatgctcctttaaracaatggcagccataagtgctatcaatttatcaccattaatggggttgccctctttgtcaacaacgc

SEQ ID NO: 29 - DNA, 179 bp, Cichorium intybus
ggaaaattcattagtttatgtgccatatttaactgctactttccatgmccacctgtatcaaatatcaactgcatggaaacatccctaaaagtatttacaatctttgtaacttttgtctattaatcacccaaaagacacaayggttgtctcatactcaactccgctttaagtcccgggaa

SEQ ID NO: 30 - DNA, 179 bp, Cichorium intybus
ggaaaattcattagtttatgtgccatatttaactgctactttccatgmccacctgtatcaaatatcaactgcatggaagcatccctaaaagtatttacaatctttgtaacttttgtctattaatcacccaaaagacacaayggttgtctcatactcaactccgctttaagtcccgggaa

SEQ ID NO: 31 - DNA, 201 bp, Cichorium intybus
ttcktttagaaacrgtaatcatgggagtatgstggcttccaccaccgttttcttatcattctgtagattgggaagtgacggttctggtggtgstggartcagagtttctttctctgacggaactggcgatgccggagtttctttctctgacggaactggcgaygccggagtttctttctttgctattggcggagttgcggc

SEQ ID NO: 32 - DNA, 201 bp, Cichorium intybus
ttcktttagaaacrgtaatcatgggagtatgstggcttccaccaccgttttcttatcattctgtagattgggaagtgacggttctggtggtgstggartcggagtttctttctctgacggaactggcgatgccggagtttctttctctgacggaactggcgaygccggagtttctttctttgctattggcggagttgcggc

SEQ ID NO: 33 - DNA, 201 bp, Cichorium intybus
tacccattgagctaaagacacatacgccacacctctytgatgacaatttggtccgaatggattatcacgagtatgacaaagtgaacctaagatagcaattaggagattacctgtgttcccacacgcagtcatgataatggtgaatcgcacaaactgtggaggcgtttacacagccamacaactataatgcccaataagaac

SEQ ID NO: 34 - DNA, 201 bp, Cichorium intybus
tacccattgagctaaagacacatacgccacacctctytgatgacaatttggtccgaatggattatcacgagtatgacaaagtgaacctaagatagcaattgggagattacctgtgttcccacacgcagtcatgataatggtgaatcgcacaaactgtggaggcgtttacacagccamacaactataatgcccaataagaac

SEQ ID NO: 35 - DNA, 201 bp, Cichorium intybus
tytaagaaagaggatccacgagatcaagatggttgagawcaactatgagccaccttctaattggatgaawtgggagaaaakatattacacaagctacgatacgtatgtttgtgaattggtgggactacttcaatcgaggttaatgaatactaggcctagtgtagctcttggtttgatggctctaatcacgttaagtttgcc

SEQ ID NO: 36 - DNA, 201 bp, Cichorium intybus
tytaagaaagaggatccacgagatcaagatggttgagawcaactatgagccaccttctaattggatgaawtgggagaaaakatattacacaagctacgatgcgtatgtttgtgaattggtgggactacttcaatcgaggttaatgaatactaggcctagtgtagctcttggtttgatggctctaatcacgttaagtttgcc

SEQ ID NO: 37 - DNA, 201 bp, Cichorium intybus
gaaaattgatdggaaactttgtgactctaggcttatcagccaaaatatttctttgaactttatctatcaatactttttgtggaggttcctctttcaaatgctcatcttcataatcattgtttacatagtatcctactcgaacaaattcttgtcccagatatgagcaygtyaatarmagractgtaacacctattatgtctt

SEQ ID NO: 38 - DNA, 201 bp, Cichorium intybus
gaaaattgatdggaaactttgtgactctaggcttatcagccaaaatatttctttgaactttatctatcaatactttttgtggaggttcctctttcaaatgttcatcttcataatcattgtttacatagtatcctactcgaacaaattcttgtcccagatatgagcaygtyaatarmagractgtaacacctattatgtctt

SEQ ID NO: 39 - DNA, 159 bp, Cichorium intybus
gtctccatggcttccatcaaaatgagaagtgtttccatcytcttcatctacattgttgctatcttaatcatgcaaatggctttgattaccgcagatcaaggaggggcattmagaacggaacgatatgatccaagaacaccaccaacaccacaaggaaac

SEQ ID NO: 40 - DNA, 159 bp, Cichorium intybus
gtctccatggcttccatcaaaatgagaagtgtttccatcytcttcatctacattgttgctatcttcatcatgcaaatggctttgattaccgcagatcaaggaggggcattmagaacggaacgatatgatccaagaacaccaccaacaccacaaggaaac

SEQ ID NO: 41 - DNA, 201 bp, Cichorium intybus
tgacygatgcaatcaaagatgctgttgggagacctcggcccgactttttctggcggtgcttccctgatggmaaaggggtttttgatcctattacaagtaacgtcatgtgtaccggattaaartccgtcatcaaagaaggacataaaagtttcccaagtgcrcacacgtcttggtcctttgctggtcttggttttctytcat

SEQ ID NO: 42 - DNA, 201 bp, Cichorium intybus
tgacygatgcaatcaaagatgctgttgggagacctcggcccgactttttctggcggtgcttccctgatggmaaaggggtttttgatcctattacaagtaatgtcatgtgtaccggattaaartccgtcatcaaagaaggacataaaagtttcccaagtgcrcacacgtcttggtcctttgctggtcttggttttctytcat

SEQ ID NO: 43 - DNA, 201 bp, Cichorium intybus
cattagctgctctattaccaaattgggctgtttactttgctgtttatgggcaactcaaagagcttcttcattcacatgccgatagcaatggccatcttagctttggtgccaacatggtagctgctgcgggtgctggtgcagccacatcaattgccacaaatcccttgtgggtwgtsaagacaagattgcaaacwcaaggaa

SEQ ID NO: 44 - DNA, 201 bp, Cichorium intybus
cattagctgctctattaccaaattgggctgtttactttgctgtttatgggcaactcaaagagcttcttcattcacatgccgatagcaatggccatcttagttttggtgccaacatggtagctgctgcgggtgctggtgcagccacatcaattgccacaaatcccttgtgggtwgtsaagacaagattgcaaacwcaaggaa

SEQ ID NO: 45 - DNA, 201 bp, Cichorium intybus
ttcctctatttgtgtctggtaaaagttgwgaaagtgcacgacatgcaccgactttaacaggggcaggcacrtccatgccgattgcctggatagcagcatacagraaatgatcaataacagcattgttgaccacagaygaraatttagcaattgatgaaaacatacgagcataaagaaagggaaattcatgtacacctgctg

SEQ ID NO: 46 - DNA, 201 bp, Cichorium intybus
ttcctctatttgtgtctggtaaaagttgwgaaagtgcacgacatgcaccgactttaacaggggcaggcacrtccatgccgattgcctggatagcagcatagagraaatgatcaataacagcattgttgaccacagaygaraatttagcaattgatgaaaacatacgagcataaagaaagggaaattcatgtacacctgctg

SEQ ID NO: 47 - DNA, 143 bp, Cichorium intybus
tttggtcccatctctctcgaacttcaacttctaatgcaaattattgtcctgtatctccctctttgagattagcatcyyaaagcagagtgacaatcaatcatttatggccaaagttcatggratcatctactcatctgtgaaaa

SEQ ID NO: 48 - DNA, 143 bp, Cichorium intybus
tttggtcccatctctctcgaacttcaacttctaatgcaaatttttgtcctgtatctccctctttgagattagcatcyyaaagcagagtgacaatcaatcatttatggccaaagttcatggratcatctactcatctgtgaaaa

SEQ ID NO: 49 - DNA, 201 bp, Cichorium intybus
cggtagatgaagcgggtgtattggtggtgagaggtgaaggtggaaaaagaatgtckaaagagaaggaaaaggagactgtttggcatgttgctgagaggggaggaggagttgacggygtgggttttttcgagggaaatcgagttaccggaggatgtgaaagttgatcagatcaaagctcaggtggaaaatggggttttgacg

SEQ ID NO: 50 - DNA, 201 bp, Cichorium intybus
cggtagatgaagcgggtgtattggtggtgagaggtgaaggtggaaaaagaatgtckaaagagaaggaaaaggagactgtttggcatgttgctgagaggggcggaggagttgacggygtgggttttttcgagggaaatcgagttaccggaggatgtgaaagttgatcagatcaaagctcaggtggaaaatggggttttgacg

SEQ ID NO: 51 - DNA, 201 bp, Cichorium intybus
tcggrgaattgtgccgttrcctgagaggaattchggtggaataaaagataaragaacaagcacacaaaggaaatggtttcgccaaatttatgaattatgtagakcaaataaaggtgatgtggtggagaatgtaggttttgccaccgcacgaacacgggataataaaaagcatattccggtggctggagagtggaattttca

SEQ ID NO: 52 - DNA, 201 bp, Cichorium intybus
tcggrgaattgtgccgttrcctgagaggaattchggtggaataaaagataaragaacaagcacacaaaggaaatggtttcgccaaatttatgaattatgtggakcaaataaaggtgatgtggtggagaatgtaggttttgccaccgcacgaacacgggataataaaaagcatattccggtggctggagagtggaattttca